(19) United States
(12) Patent Application Publication
(10) Pub. No.: US 2026/0365553 A1
(43) Pub. Date: Nov. 19, 2026

(54) ACTIVATED SLUDGE AND PREPARATION METHOD FOR TREATING COMPOUND POLLUTION WASTEWATER CONTAINING ARSENATE AND BUTYL XANTHATE

(71) Applicant: South China Normal University, Guangzhou (CN)

(72) Inventors: Bo Yan, Guangzhou (CN); Jiayi Zhou, Guangzhou (CN); Zemin Li, Guangzhou (CN); Lijuan Zhang, Guangzhou (CN); Xipeng Wei, Guangzhou (CN)

(21) Appl. No.: 19/420,844

(22) Filed: Dec. 16, 2025

(30) Foreign Application Priority Data
May 15, 2025 (CN) 202510622824.0

(51) Int. Cl. C02F 3/34; C02F 3/28; C02F 101/10; C02F 101/34; C12N 1/20

(52) U.S. Cl. CPC C02F 3/341; C02F 3/28; C12N 1/20; C02F 2101/103; C02F 2101/34; C02F 2101/40

(57) ABSTRACT

An activated sludge for treating compound pollution wastewater containing arsenate and butyl xanthate, and a preparation method thereof are provided. The preparation method includes: step 1: taking soil near lead-zinc tailings as an original sample to obtain an initial sludge; step 2, preparing a culture medium for cultivating sludge; step 3, adding culture medium to an anaerobic flask, and then putting the initial sludge into the flask; step 4, adding sodium arsenate and butyl xanthate to the flask, stirring evenly, releasing oxygen, shaking and cultivating to obtain domesticated anaerobic activated sludge. The present disclosure uses specific collection and domestication steps at specific locations to cultivate activated sludge with unique biological characteristics. It can not only reduce arsenate without the addition of additional organic carbon sources, but also effectively oxidize and remove xanthate, meeting the low-cost and high-efficiency requirements for removing arsenate and xanthate from mineral processing wastewater.

### CROSS-REFERENCE TO RELATED APPLICATIONS

This application claims priority to Chinese Patent Application No. 202510622824.0, filed on May 15, 2025, which is hereby incorporated by reference in its entirety.

### TECHNICAL FIELD

The present disclosure relates to the field of wastewater treatment technologies, and in particular, to an activated sludge and a preparation method for treating compound pollution wastewater containing arsenate and butyl xanthate.

### BACKGROUND

Arsenic (As) is a highly toxic heavy metal element that is widely present in both natural and anthropogenic environments. Its natural sources mainly include rock weathering, volcanic activity, and natural release from soil. In human activities, arsenic mainly comes from the mining and smelting processes of non-ferrous metal deposits. For example, arsenic is often associated with non-ferrous metal deposits such as gold, copper, nickel, cobalt, etc. During a smelting process, arsenic will enter the cigarette ash or wet smelting slag, thereby forming arsenic containing waste. Besides that, a production process of semiconductor materials such as gallium arsenide in the electronics industry also generates arsenic containing wastewater. The discharge of arsenic containing wastewater poses a serious threat to the environment and human health. Arsenic and its compounds are classified as a class of carcinogens by the International Agency for Research on Cancer (IARC), with the “triple effect” of mutagenicity, teratogenicity, and carcinogenesis. In industrial wastewater, arsenic mainly exists in forms of As (III) (trivalent arsenic) and As (V) (pentavalent arsenic). If arsenic containing wastewater is discharged directly without treatment, it can lead to water pollution, soil arsenic enrichment, and crop arsenic accumulation, thereby threatening human health through the food chain.

In mining activities, flotation wastewater is one of the important sources of pollution generated during the beneficiation process. The commonly used collector in a flotation process, xanthate (alkyl dithiocarbonate), is the main harmful component of flotation wastewater. Xanthate has high toxicity, irritating odor, and is difficult to degrade in water. A small amount can cause the water to become foul smelling and spoiled. According to statistics, the annual discharge of mining wastewater in China is huge, with the discharge of mineral processing wastewater being particularly prominent. It is expected that its consumption will reach about 370 million tons by 2025. A large amount of untreated wastewater is directly discharged, which caused serious pollution to water body and surrounding ecological environment.

In the published Chinese patents, there are relatively few studies and reports on the efficient removal of butyl xanthate and heavy arsenate from flotation wastewater by biological methods, mainly focusing on the use of a single strain for action, and generally only able to remove butyl xanthate or arsenate. There are no relevant research and report on the treatment method of arsenate and butyl xanthate compound polluted wastewater by microorganisms in activated sludge.

In terms of removing butyl xanthate and heavy metals, CN109574407A discloses a rapid degradation of butyl xanthate in flotation wastewater using bacterium Shewanella oneidensis MR-1 under aerobic conditions. Under the anaerobic conditions, Cr (VI) is used as an electron acceptor to immobilize and harmlessly remove Cr (VI) from the flotation wastewater, thereby achieving efficient and rapid removal of butyl xanthate and heavy metal Cr (VI) from the flotation wastewater. CN115010302A discloses selective degradation and removal of some heavy metals and xanthate flotation agents with poor flotation selectivity in lead-zinc flotation wastewater by regulating an input voltage of an electric field outside the electrocatalytic internal electrolysis reactor. CN111018085A discloses a method for treating heavy metal flotation wastewater containing xanthate using chemical oxidation, which achieves a degradation of xanthate in flotation wastewater and synchronous deep removal of heavy metals in the flotation wastewater. The acidophilic elemental sulfur reducing bacteria disclosed in CN113748091A use organic carbon to reduce elemental sulfur to sulfides. Pentavalent arsenic is reduced to trivalent arsenic by the combined action of elemental sulfur reducing bacteria with arsenic reducing ability and sulfides. Trivalent arsenic and sulfides form arsenic sulfur compound precipitates, thereby rapidly and synchronously removing trivalent arsenic and pentavalent arsenic from wastewater.

In summary, existing treatment technologies can only remove arsenate or xanthate separately, or use a single bacterial strain or chemical oxidation method to remove xanthate and heavy metals from the flotation wastewater, and the heavy metals do not include arsenic. With an acceleration of industrialization, the treatment of arsenate containing wastewater and flotation wastewater containing xanthate has become an urgent environmental problem to be solved. Chemical precipitation, adsorption, and oxidation methods still have shortcomings in removal efficiency and economy, and may generate secondary pollution. Biological methods are an efficient, economical, and environmentally friendly treatment technology, and compared to using a single strain, activated sludge does not require screening, separation, purification, and addition of organic carbon sources. Therefore, it has important practical significance for solving the pollution problem of flotation wastewater containing arsenate and xanthate.

### SUMMARY

The purpose of the present disclosure is to provide a method for treating wastewater containing arsenate and butyl xanthate compound pollution based on microorganisms in activated sludge, which can simultaneously achieve the biological characteristics of xanthate oxidation removal and arsenate reduction fixation without an addition of additional organic carbon sources. The present disclosure further provides an application using this method, which has good removal efficiency, simple operation, and can meet needs of low-cost and efficient removal of arsenate and xanthate from the flotation wastewater. It has good application prospects in mining or other oligotrophic environments.

The purpose of the present disclosure is achieved through the following technical solutions.

In a first aspect, the present disclosure provides a method for preparing activated sludge for treating compound pollution wastewater containing arsenate and butyl xanthate, including the following steps:
step 1: taking soil near lead-zinc tailings as an original sample to obtain an initial sludge;
step 2, preparing a culture medium for cultivating sludge;
step 3, adding culture medium to an anaerobic flask, and then putting the initial sludge into the flask;
step 4, adding sodium arsenate and butyl xanthate to the flask, stirring evenly, releasing oxygen, shaking and cultivating to obtain domesticated anaerobic activated sludge.

In an implementation mode, in step 1, a sample collection uses a plum blossom point sampling method for fixed-point sampling, and surface, middle, and deep soil are collected from the soil near a tailings discharge outlet of mine and stored in sterile sampling bags for refrigerated transportation and backup.

In an implementation mode, in step 1, an initial microbial community in the soil near the lead-zinc tailings includes: Pseudomonadota 75.77%, Bacillota 9.41%, Nitrospirota 3.54%, Deinococcota 2.84%, Actinomycetota 1.22%, Dependentiae 1.01%, and other microorganisms 6.21%.

In an implementation mode, in step 2, components in the culture medium are calculated by concentration, including CaCl₂ of 0.2460 g/L, MgCl₂ of 1.0572 g/L, NaCl of 0.4459 g/L, KH₂PO₄ of 0.0299 g/L, KCl of 0.0283 g/L, NaHCO₃ of 0.8082 g/L, and NH₄Cl of 0.1557 g/L.

In an implementation mode, in step 3, pH is adjusted to 7.0±0.1, and an initial sludge concentration is 3.0 g/L.

In an implementation mode, in step 4, a concentration of sodium arsenate added is 0.01-0.03 g/L, and a concentration of butyl xanthate added is 0.1-0.3 g/L.

In an implementation mode, in step 4, the oxygen is discharged by introducing pure nitrogen gas into the anaerobic flask for at least 20 minutes.

In an implementation mode, in step 4, a temperature for oscillation cultivation is 25 °C, a speed is 150-180 r/min, and a cultivation time is 10-14 days.

In an implementation mode, in step 4, microbial community of the anaerobic activated sludge includes: Pseudomonadota 90.80%, Bacteroidota 6.14%, Dependentiae 2.41%, and other microorganisms 0.65%.

In a second aspect, the present disclosure provides an activated sludge for treating compound pollution wastewater containing arsenate and butyl xanthate, where the activated sludge is prepared using the preparation method.

In a third aspect, the present disclosure provides an application of the activated sludge for treating compound pollution wastewater containing arsenate and butyl xanthate.

The beneficial effects of the present disclosure are as follows.

1. The present disclosure uses specific collection and domestication steps at specific locations to cultivate activated sludge with unique biological characteristics. It can not only reduce arsenate without the addition of additional organic carbon sources, but also effectively oxidize and remove xanthate, thereby meeting the low-cost and high-efficiency requirements for removing arsenate and xanthate from mineral processing wastewater in low nutrient mining areas.

2. The activated sludge prepared by the method of the present disclosure is dominated by 90.80% Pseudomonadota and 6.14% Bacteroidota as dominant bacterial genera. This genus of bacteria was identified from tailings samples and has strong adaptability to oligotrophic mining environments (arsenate pollution). Its application in bioremediation can reduce environmental remediation costs. And through the screening and domestication of dominant bacterial strains, microbial agents with specific functions can be developed for use in environmental protection, industrial production, and other fields.

3. The method of the present disclosure is applied to the treatment of wastewater containing arsenate and butyl xanthate compound pollution. Under anaerobic conditions, microorganisms in the activated sludge can oxidize butyl xanthate and use its sulfur source to reduce and fix arsenate. In 7 days, the removal rate of 50 mg/L butyl xanthate was 99.96%, corresponding to a TOC utilization rate of 80.4%, the As (V) reduction rate of 72.93%, and a TAs removal rate of 65.26%.

4. The method of the present disclosure was applied to the treatment of continuous flow wastewater containing arsenate and butyl xanthate compound pollution. Under anaerobic conditions, the removal rate of 200 mg/L butyl xanthate was 99.64%, the As (V) reduction rate was 96.13%, and the TAs removal rate was 94.70% in 30 days.

5. The method of the present disclosure can better utilize xanthate as an organic substrate, maintain a high removal rate of xanthate, and achieve the removal of organic carbon and arsenate. Applying this method to the treatment of wastewater containing xanthate and arsenate in mining areas is beneficial for simplifying the wastewater treatment process, improving treatment efficiency, meeting environmentally friendly requirements, and having good economic and environmental benefits. It can overcome the problem of difficult biological growth caused by insufficient carbon sources, rendering it possible to simultaneously remove xanthate and arsenate, and has broad application prospects.

### BRIEF DESCRIPTION OF DRAWINGS

The present disclosure is further illustrated using the accompanying drawings, but the embodiments shown in the drawings do not constitute any limitation on the present disclosure. For those skilled in the art, other drawings can be obtained based on the following drawings without creative work.

FIG. 1 shows a relative abundance of microbial community composition between an original tailings sample (a) and a domesticated activated sludge (b) in Example 1 of the present disclosure.

FIG. 2 shows a removal effect of the original tailings sample (front) and the activated sludge (after) obtained after 14 days of domestication on wastewater containing butyl xanthate (A₃₀₁) and arsenate in Example 1 of the present disclosure.

FIG. 3 shows a removal effect of butyl xanthate (BX) and total organic carbon (TOC) on wastewater containing butyl xanthate and arsenate under anaerobic conditions using tailings samples from Example 2 of the present disclosure.

FIG. 4 shows an arsenate removal effect of tailings samples from Example 2 of the present disclosure on wastewater containing butyl xanthate and arsenate under anaerobic conditions.

FIG. 5 shows a removal efficiency of tailings samples from Example 2 of the present disclosure under anaerobic conditions for wastewater containing butyl xanthate and arsenate.

FIG. 6 shows a scanning electron microscope (SEM) image of the sludge solid sample from Example 3 of the present disclosure under anaerobic conditions.

FIG. 7 shows an X-ray diffraction (XRD) pattern of the solid sludge sample from Example 3 of the present disclosure under anaerobic conditions.

FIG. 8 shows the removal effect of butyl xanthate (BX) and arsenate from wastewater containing butyl xanthate and arsenate under anaerobic and continuous flow conditions using tailings samples from Example 4 of the present disclosure.

### DESCRIPTION OF EMBODIMENTS

The following provides specific examples to illustrate the technical solution of the present disclosure. It should be understood that the one or more method steps mentioned in the present disclosure do not exclude the existence of other method steps before and after the combination step or the insertion of other method steps between these explicitly mentioned steps. It should also be understood that these embodiments are only used to illustrate the present disclosure and not to limit the scope of the present disclosure. Moreover, unless otherwise specified, the numbering of each method step is only convenient for identifying each method step, and is not intended to limit the order of arrangement of each method step or limit the scope of the present disclosure that can be implemented. Any changes or adjustments in their relative relationships, without substantial changes in the technical solution, shall also be considered as within the scope of the present disclosure that can be implemented.

In order to better understand the above technical solutions, the following provides a more detailed description of exemplary embodiments of the present disclosure. Although exemplary embodiments of the present disclosure have been shown, it should be understood that the present disclosure may be implemented in various forms and should not be limited by the embodiments described herein. On the contrary, these embodiments are provided to enable a more thorough understanding of the present disclosure and to fully convey the scope of the present disclosure to those skilled in the art.

The present disclosure will be further described in combination with the following embodiments.

### EXAMPLE 1

A process for treating activated sludge from wastewater contaminated with arsenate and butyl xanthate, including:

### Step 1, Collection Process of Activated Sludge

An original sample of the present disclosure was collected near the discharge outlet of lead-zinc tailings in Renhua District, Shaoguan City, Guangdong Province, and then domesticated and cultivated. The sample collection was carried out according to the “Mixed Sample Collection Method” in the “Technical Specifications for Soil Environmental Monitoring” (HJ/T 166-2004). A plum blossom sampling method was used for fixed-point sampling, and surface, middle, and deep soil samples were collected from the soil near a tail outlet and stored in sterile sampling bags at 4 °C for refrigeration and storage.

Step 2, Cultivation and domestication process of activated sludge

### (1) Preparation of Activated Sludge Culture Medium

CaCl₂ 0.2460 g/L, MgCl₂ 1.0572 g/L, NaCl 0.4459 g/L, KH₂PO₄ 0.0299 g/L, KCl 0.0283 g/L, NaHCO₃ 0.8082 g/L, NH₄Cl 0.1557 g/L.

### (2) Sludge Mixing

Adding 150 mL of the culture medium prepared in (1) to an anaerobic flask, and then adding mine tailings sludge, sodium arsenate, and butyl xanthate to the flask respectively. After addition, corresponding concentrations of activated sludge, sodium arsenate, and butyl xanthate in the anaerobic flask are 3 g/L, 0.02 g/L, and 0.2 g/L.

### (3) Sludge Domestication

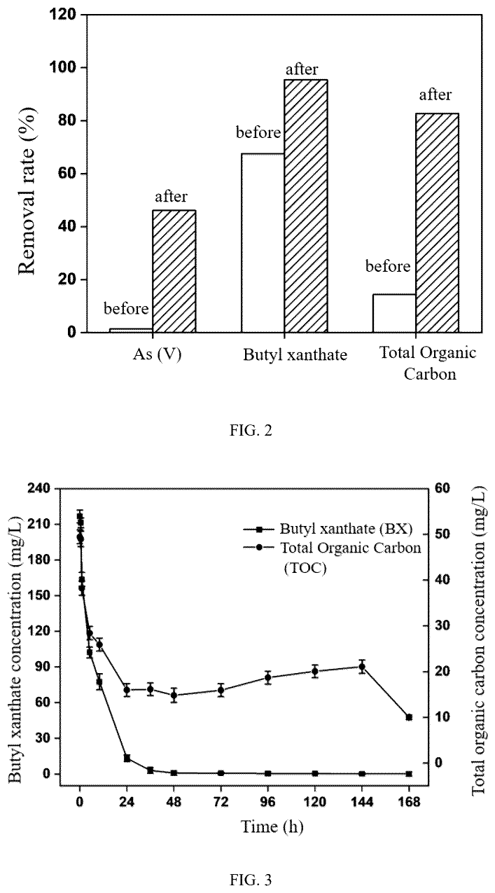

After stirring evenly, pure nitrogen gas was introduced into the anaerobic flask for 20 minutes. The fixed domestication conditions were pH=7.0, 25 °C, and 160 rpm/min shaking culture for 10-14 days to obtain anaerobic activated sludge. Where the pH value is adjusted by 1 mol/L hydrochloric acid and sodium hydroxide solution. The results of domestication for 14 days are shown in FIG. 2. After domestication, the removal rates of butyl xanthate and corresponding TOC and As(V) by activated sludge were 95.45%, 82.71%, and 46.07%, respectively, which showed significant improvement in removal ability compared to the original sample.

Step 3, 16S Amplicon Sequencing Analysis

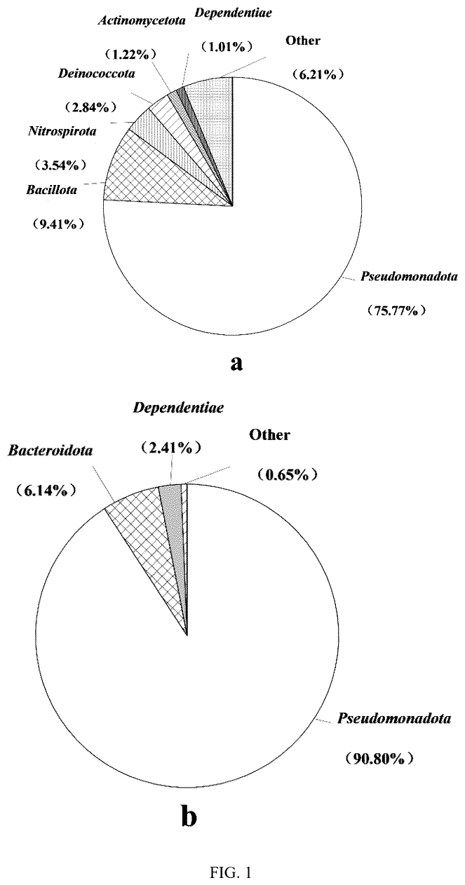

16S amplicon analysis was performed on the original sludge sample and domesticated sludge sample, and sequencing was completed by Shanghai Bioengineering Co., Ltd. As shown in FIG. 1, the composition and relative abundance of microbial communities can be determined. The initial sample was activated sludge with Pseudomonadota (75.77%) and Bacillota (9.41%) as a dominant bacterial genera. The domesticated samples were anaerobic activated sludge dominated by 90.80% Pseudomonadota and 6.14% Bacteroidota.

### EXAMPLE 2

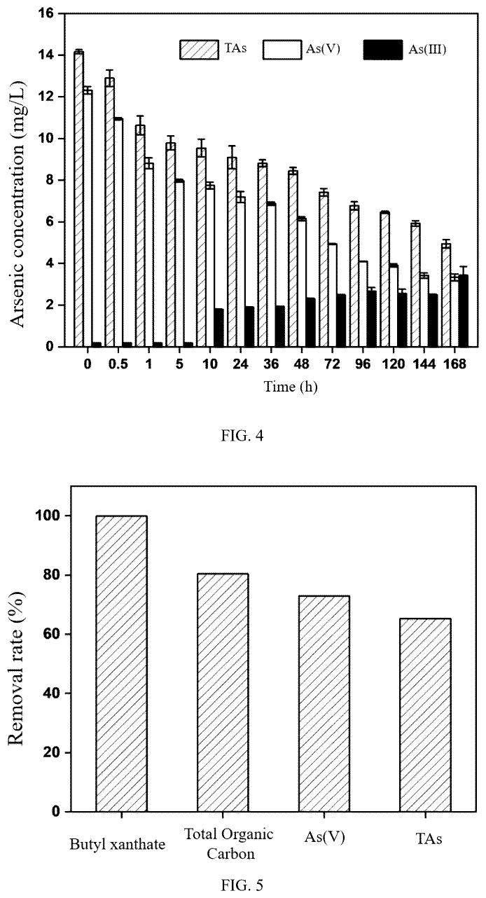

This embodiment provides an application of the activated sludge on the basis of Example 1, and applies it to the biological treatment of wastewater containing arsenate and butyl xanthate compound pollution. The results are shown in FIGS. 3-5 in the attached figures.

### A Specific Process is as Follows

Adding 150 mL of the activated sludge culture medium prepared in Example 1 and the successfully domesticated anaerobic activated sludge to the anaerobic flask, followed by an addition of sodium arsenate and butyl xanthate. concentrations of activated sludge, sodium arsenate, and butyl xanthate in the anaerobic flask after addition are 3 g/L, 0.02 g/L, and 0.05 g/L. After stirring evenly, pure nitrogen gas was introduced into the anaerobic flask for 5 minutes. The fixed cultivation conditions were 25 °C, 160 rpm/min, pH=7.0, and shaken for 7 days.

Culture media were collected at 0 h, 0.5 h, 1 h, 5 h, 10 h, 24 h, 36 h, 48 h, 72 h, 96 h, 120 h, and 144 h, and centrifuged at low speed for 5-10 min at 5000 r/min and 4 °C. A supernatant was filtered through 0.22 μm membranes and the TOC (total organic carbon), A₃₀₁ (butyl xanthate), As(III) , As(V)⁺, and TAs (total arsenic) contents were measured.

Three experimental groups with repeated techniques and a blank control group were set up in the experiment, with the control group receiving an equal amount of physiological saline.

From this embodiment and FIGS. 3-5 and FIGS. 1-2 of example 1, it can be seen that the activated sludge can utilize the organic carbon source in butyl xanthate to grow, reduce the content of xanthate, and achieve the reduction of As(V) and removal of TAs during the process. Under anaerobic conditions, the removal rate of butyl xanthate was 99.87%, corresponding to a removal rate of 80.4% for TOC, 72.93% for As (V), and 65.26% for TAs (total arsenic).

### EXAMPLE 3

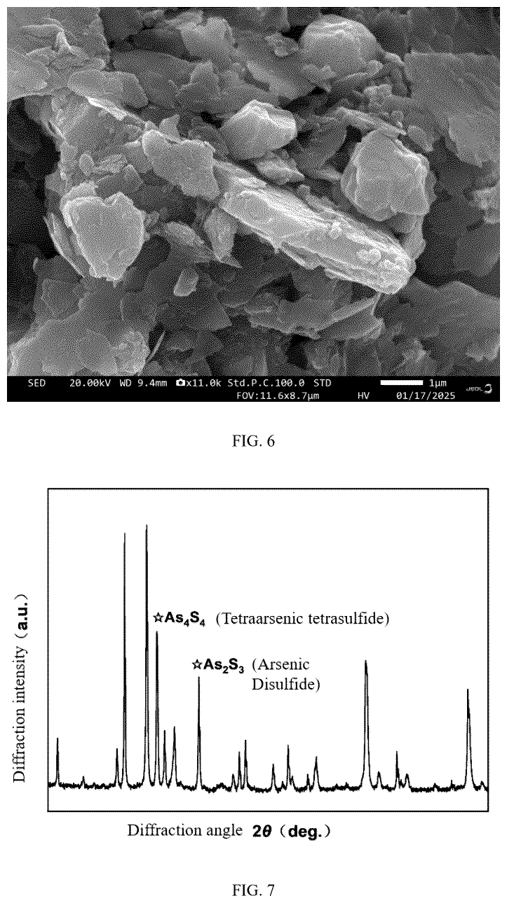

This embodiment is based on Example 2, and the performance characterization of the activated sludge after treatment method is carried out. The specific projects include SEM (scanning electron microscopy) and XRD (X-ray diffraction), which were completed by Hunan Nano Micro New Material Technology Co., Ltd., as shown in FIGS. 6 and 7.

The activated sludge treated with the wastewater containing butyl xanthate and arsenate compound pollution in Example 2 was characterized. The SEM (scanning electron microscope) results are shown in FIG. 6. The surface of the activated sludge presents irregular block structures, which are relatively rough and have a large specific surface area. It may have high reactivity for the treatment of wastewater with compound pollution. The XRD (X-ray diffraction) results are shown in FIG. 7, located at 2θ=29.42° and 33.02° (marked in the figure), belonging to the As₄S₄ and As₂S₃ phases, respectively. Under anaerobic conditions, there are many particles attached to a surface of the activated sludge, but the size is small. It is preliminarily judged that sulfur-containing and arsenate compounds are generated on the activated sludge.

### EXAMPLE 4

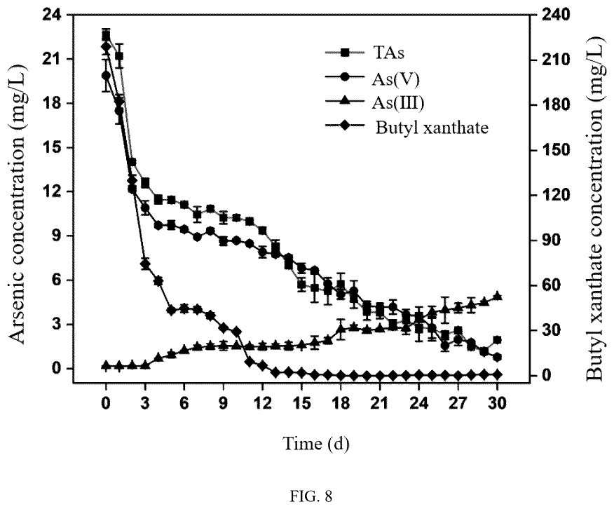

This embodiment provides the application of the treatment method based on Examples 1 and 2, and applies it to the biological treatment of continuous flow wastewater containing arsenate and butyl xanthate compound pollution, as shown in FIG. 8.

The treatment process of wastewater containing arsenate and butyl xanthate compound pollution by the method is as follows.

Adding successfully domesticated anaerobic activated sludge to a reactor, preparing 35 L of culture medium according to the method in Example 1, and adding 2.915 g of disodium arsenate heptahydrate and 7 g of butyl xanthate. Storing in the inlet tank and continuously pump a solution into the reactor at a flow rate of 0.8 mL/min using a peristaltic pump. The concentrations of anaerobic activated sludge, sodium arsenate, and butyl xanthate after domestication in Example 1 of the reactor were 3 g/L, 0.02 g/L, and 0.2 g/L, respectively. The reaction conditions for continuous flow are: hydraulic retention time (HRT) set to 24 hours, and dissolved oxygen (DO) controlled between 0.5-1.0 mg/L through a nitrogen aeration device. The pH value was adjusted to between 7.0±1.0 using NaHCO₃, and the temperature was kept at room temperature for continuous cultivation for 30 days. Samples were taken once a day (d), and the samples were centrifuged at low speed for 5-10 minutes at 5000 r/min and 4 °C in a freeze centrifuge. The supernatant was filtered through 0.22 μm membranes and the contents of A₃₀₁ (butyl xanthate), As(III) , As(V) , and TAs were measured.

From this example and FIG. 8, it can be seen that under continuous flow cultivation conditions, the activated sludge can remove high concentrations of xanthate and reduce and remove arsenate more efficiently than the activated sludge grown within 7 days (Example 2). Under anaerobic conditions, 200 mg/L of butyl xanthate can be completely removed before 14 days, with a final removal rate of 99.64%. Arsenate rapidly decreases in the first 3 days, and is continuously and stably reduced and removed in a later process. The As (V) reduction rate is 96.13%, and the TAs removal rate is 94.70%.

Based on the above embodiments, the treatment method provided by the present disclosure can be widely applied in the field of treating composite polluted wastewater from mines containing arsenate and butyl xanthate. The activated sludge in the method of the present disclosure is composed of Pseudomonadota (90.80%) and Bacteroidota (6.14%) as the dominant bacterial genera. It has strong adaptability to arsenate polluted environments, can reduce arsenate without adding additional organic carbon sources, and can effectively oxidize and remove xanthate, meeting the low-cost and efficient removal needs of arsenate and xanthate in mineral processing wastewater such as oligotrophic mining areas. Under anaerobic conditions, microorganisms in the activated sludge can oxidize butyl xanthate and use its C source to reduce and fix arsenate. Within 7 days, the removal rate of 50 mg/L butyl xanthate was 99.96%, corresponding to a TOC (total organic carbon) utilization rate of 80.4%, As (V) reduction rate of 72.93%, and TAs (total arsenate) removal rate of 65.26%. Under continuous flow conditions, the removal rate of 200 mg/L butyl xanthate within 30 days was 99.64%, the As (V) reduction rate was 96.13%, and the TAs (total arsenic) removal rate was 94.70%.

Applying this method to the treatment of wastewater containing xanthate and arsenate in mining areas is beneficial for simplifying the wastewater treatment process, improving treatment efficiency, meeting environmentally friendly requirements, and having good economic and environmental benefits. It can overcome the problem of difficult biological growth caused by insufficient carbon sources, rendering it possible to simultaneously remove mineral processing agents such as xanthate and arsenate, and have broad application prospects.

In the description of this specification, the reference to terms “one embodiment”, “some embodiments”, “examples”, “specific examples”, or “some examples” means that the specific features, structures, materials, or characteristics described in combination with the embodiment or example are included in at least one embodiment or example of the present disclosure. In this specification, the schematic expressions of the above terms should not be understood as necessarily referring to the same embodiments or examples. Moreover, the specific features, structures, materials, or characteristics described can be combined in any one or more embodiments or examples in a suitable manner. Besides that, those skilled in the art can join and combine different embodiments or examples described in this specification.

Although the embodiments of the present disclosure have been shown and described above, it can be understood that the above embodiments are exemplary and should not be construed as limiting the present disclosure. Those skilled in the art can make changes, modifications, substitutions, and variations to the above embodiments within the scope of the present disclosure.

What is claimed is:

1. A method for preparing activated sludge for treating compound pollution wastewater containing arsenate and butyl xanthate, comprising the following steps:
step 1: taking soil near lead-zinc tailings as an original sample to obtain an initial sludge;
step 2, preparing a culture medium for cultivating sludge;
step 3, adding culture medium to an anaerobic flask, and then putting the initial sludge into the flask;
step 4, adding sodium arsenate and butyl xanthate to the flask, stirring evenly, releasing oxygen, shaking and cultivating to obtain domesticated anaerobic activated sludge;
wherein in step 4, microbial community of the anaerobic activated sludge comprises: Pseudomonadota 90.80%, Bacteroidota 6.14%, Dependentiae 2.41%, and other microorganisms 0.65%.

2. The method for preparing activated sludge for treating compound pollution wastewater containing arsenate and butyl xanthate according to claim 1, wherein in step 1, a sample collection uses a plum blossom point sampling method for fixed-point sampling, and surface, middle, and deep soil are collected from the soil near a tailings discharge outlet of mine and stored in sterile sampling bags for refrigerated transportation and backup.

3. The method for preparing activated sludge for treating compound pollution wastewater containing arsenate and butyl xanthate according to claim 1, wherein an initial microbial community in the soil near the lead-zinc tailings comprises: Pseudomonadota 75.77%, Bacillota 9.41%, Nitrospirota 3.54%, Deinococcota 2.84%, Actinomycetota 1.22%, Dependentiae 1.01%, and other microorganisms 6.21%.

4. The method for preparing activated sludge for treating compound pollution wastewater containing arsenate and butyl xanthate according to claim 1, wherein in step 2, components in the culture medium are calculated by concentration, comprising CaCl₂ of 0.2460 g/L, MgCl₂ of 1.0572 g/L, NaCl of 0.4459 g/L, KH₂PO₄ of 0.0299 g/L, KCl of 0.0283 g/L, NaHCO₃ of 0.8082 g/L, and NH₄Cl of 0.1557 g/L.

5. The method for preparing activated sludge for treating compound pollution wastewater containing arsenate and butyl xanthate according to claim 1, wherein in step 3, pH is adjusted to 7.0±0.1, and an initial sludge concentration is 3.0 g/L.

6. The method for preparing activated sludge for treating compound pollution wastewater containing arsenate and butyl xanthate according to claim 1, wherein in step 4, a concentration of sodium arsenate added is 0.01-0.03 g/L, and a concentration of butyl xanthate added is 0.1-0.3 g/L.

7. The method for preparing activated sludge for treating compound pollution wastewater containing arsenate and butyl xanthate according to claim 1, wherein in step 4, pure nitrogen gas is introduced into an anaerobic flask for at least 20 minutes to discharge oxygen; a temperature for oscillation cultivation is 25 °C, a speed is 150-180 r/min, and a cultivation time is 10-14 days.

8. An activated sludge for treating compound pollution wastewater containing arsenate and butyl xanthate, wherein the activated sludge is prepared using the preparation method according to claim 1.

9. An application of the activated sludge according to claim 8 for treating compound pollution wastewater containing arsenate and butyl xanthate.
